(19) United States
(12) Patent Application Publication
(10) Pub. No.: US 2009/0254126 A1
(43) Pub. Date: Oct. 8, 2009

(54) COMPRESSION/DISTRACTION OSTEOTOMY SYSTEM, PLATE, METHOD, DRILL GUIDE AND SAW GUIDE

(75) Inventors: Jorge L. Orbay, Miami, FL (US); Thomas H. Norman, Miami, FL (US); William Garcia De Quevedo, Miami, FL (US)

Correspondence Address: LERNER GREENBERG STEMER LLP, P O BOX 2480, HOLLYWOOD, FL, 33022-2480, US

(73) Assignee: SKELETAL DYNAMICS LLC, Miami, FL (US)

(21) Appl. No.: 12/418,281

(22) Filed: Apr. 3, 2009

Related U.S. Application Data
(60) Provisional application No. 61/042,423, filed on Apr. 4, 2008.

(51) Int. Cl. A61B 17/66; A61B 17/80; A61B 17/86; A61B 17/58; A61F 5/00

(52) U.S. Cl. 606/282; 606/286; 606/301; 606/96; 606/87

(57) ABSTRACT

An osteotomy system includes anchoring screws and advancement screws having a head with a surface defining a first angled plane. A plate has anchoring holes and advancement holes with countersunk surfaces defining respective second angled planes for compression or distraction. The first angled plane contacts one of the second angled planes upon tightening an advancement screw, causing advancement of the plate and step compression or distraction of one bone segment relative to another. In another embodiment, a wedge has a surface defining a first angled plane. A plate has one portion with inclined surfaces defining second angled planes. An advancement screw tightens the first angled plane against one of the second angled planes to advance one of the plate body portions for compression or distraction of one bone segment attached thereto relative to another segment. A plate, a method, a drill guide and a saw guide are also provided.

### REFERENCE TO RELATED APPLICATION

The present application claims priority to the co-pending Provisional Patent Application No. 61/042,423, filed on Apr. 4, 2008 and entitled “Compression/Distraction Osteotomy System, Plate, Method and Drill Guide”, which application is being incorporated herein, by reference, in its entirety.

### BACKGROUND OF THE INVENTION

1. Field of the Invention

The invention relates to an osteotomy system and plate, as well as a method for operating the system, the plate, a drill guide and a saw guide. Osteotomy is used, in particular, to surgically divide or section bone, reposition the bone segments, and then stabilize the segments by attaching a bone plate. In certain cases, the osteotomy may be performed at an oblique angle (oblique osteotomy). Typical osteotomy procedures are used in the compression of the ulna bone and distraction of the radius bone to correct deformities of the upper extremity.

2. Description of the Related Art

Some current devices achieve compression or distraction by the use of cumbersome compression or distraction assemblies which are attached to the bone plate during the procedure, and are subsequently removed upon securing the plate to the bone. Other existing devices are capable of achieving compression of two bone segments by use of eccentrically drilled screw holes providing what is called DCP (Dynamic Compression Plate) action, but available DCP technology cannot reposition the bone segments beyond 1-2 mm.

U.S. Patent Application Publication No. US 2005/0277941 discloses a method and device for use in osteotomy, in which a plate is placed on bone segments with screws and nails. Pliers are used for compression of the bone segments by attaching one leg of the pliers to a hole in the plate and placing another leg of the pliers behind two nails or around a screw or around an additional nail. The device is cumbersome and requires a large incision since much of the device is located outside the body.

U.S. Pat. No. 4,929,247 discloses a bone compression and distraction device having an adjustment assembly with compression screws and distraction screws which pass through blocks above a plate. That device is also cumbersome and requires the blocks and screws to be placed outside of the body, once again requiring a large wound.

U.S. Pat. No. 4,705,031 discloses an osteosynthesis plate for the pressure stabilization of bone fragments or segments. The plate has a plurality of elongated slots with ramped hole sections to be placed at each of the bone segments. The ramped hole sections at each side of the plate have ramps inclined in one direction on one side and in the opposite direction on the other side of the plate. Screws are alternately tightened and loosened along the ramps for displacing and compressing the bone segments. A different plate, where the ramp arrangement is inverted is required to achieve distraction.

U.S. Pat. No. 3,552,389 shows an osteosynthetic pressure plate construction in which a plate is placed on a bone. The plate is machined with multiple tools to form slots each having a ramp on only one side. A screw is tightened in a slot in such a way that a seating surface of the head of the screw slides along the ramp and compresses the bone segments together. The slots may be formed for spherical or conical screw heads, but in each case the ramp is formed on one side of the slot only, which permits movement in only one direction.

Therefore, even those prior art devices that are not as cumbersome do not permit both compression and distraction using the same plate, but rather, require a different plate for each function.

### SUMMARY OF THE INVENTION

It is accordingly an object of the invention to provide an osteotomy system, plate, method, drill guide and saw guide, which overcome the hereinafore-mentioned disadvantages of the heretofore-known devices and methods of this general type and which eliminate the need for additional devices, simplifying and speeding the surgical procedure and therefore reducing patient risk, as well as decreasing medical costs. More specifically, it is preferred that the device should not be cumbersome, should be almost entirely disposed within the body, should permit one plate to perform compression or distraction, should allow screws to be easily placed in a desired location and should allow for a variety of differently shaped planes resulting in different degrees of movement when contacted by screws.

With the foregoing and other objects in view there is provided, in accordance with the invention, a step compression/distraction osteotomy system. Step compression/distraction is defined as sequential incremental advancement of the bone plate to either close or open the gap between the bone segments created by the osteotomy procedure. The system comprises a plurality of anchoring screws each having screw threads and a screw head as well as a plurality of advancement screws each having screw threads and a screw head with a surface defining a first angled plane. A plate has a plate body with a distal end and a proximal end. The plate body has anchoring holes formed therein for receiving the anchoring screws to fasten the plate to a first bone segment. The plate body also has a longitudinal slot formed therein and a series of advancement holes formed therein and merging into each other along the longitudinal slot. The advancement holes each have a countersunk surface forming a seat for receiving advancement screws to be screwed into a second bone segment. The countersunk surfaces each define one second angled plane facing the distal end and another second angled plane facing the proximal end wherein the surface of each second angled plane allows tangential, sliding contact by the first angled plane of an advancement screw head. The surface defining the first angled plane of the advancement screw head contacts the one second angled plane for step compression or the other second angled plane for step distraction of the first bone segment relative to the second bone segment by advancement of the plate upon one of the advancement screws being screwed into holes tapped into the second bone segment.

Although the longitudinal slot is described in connection with the preferred embodiment, it may also be omitted, if desired, and the plate may be anchored in ways other than with anchoring screws. In addition, the one second angled plane and the other second angled plane may be disposed at different angles within at least one of the countersunk surfaces and spaced apart differently, for compression and distraction by different amounts.

With the objects of the invention in view, there is also provided a step compression/distraction osteotomy plate. The plate comprises a plate body with a distal end, a proximal end, and anchoring holes for receiving anchoring screws to fasten the plate to a first bone segment. The plate body also has a longitudinal slot and a series of advancement holes merging into each other along the longitudinal slot. The advancement holes each have a countersunk surface forming a seat for receiving advancement screws having a screw head defining a first angled plane. If desired, the anchoring holes and advancement holes may be configured as slotted keyholes, as will be more fully described herebelow. The countersunk surfaces of the advancement holes each define one second angled plane facing the distal end and another second angled plane facing the proximal end, with the surface of each second angled plane of an advancement hole allowing tangential, sliding contact by the first angled plane of an advancement screw head. In particular, the one second angled plane of an advancement hole may be contacted by a first angled plane of an advancement screw head for step compression or the other second angled plane of the advancement hole may be contacted by a first angled plane of an advancement screw head for step distraction of the first bone segment relative to a second bone segment by advancement of the plate when an advancement screw is screwed into holes tapped into the second bone segment.

Once again, the longitudinal slot of the presently described preferred embodiment may be omitted, if desired, and the plate may be anchored in ways other than with anchoring screws. The one second angled plane and the other second angled plane may be disposed at different angles within at least one of the countersunk surfaces and spaced apart differently, for compression and distraction by different amounts, as mentioned above.

With the objects of the invention in view, there is additionally provided a step compression/distraction osteotomy method. In one particular embodiment, the method comprises providing a plurality of anchoring screws each having screw threads and a screw head; a plurality of advancement screws each having screw threads and a screw head with a surface defining a first angled plane; a plate having a plate body with a distal end and a proximal end; anchoring holes in the plate body for receiving the anchoring screws to fasten the plate to a first bone segment, a longitudinal slot in the plate body and a series of advancement holes in the plate body merging into each other along the longitudinal slot. The advancement holes each have a countersunk surface defining one second angled plane facing the distal end and another second angled plane facing the proximal end, wherein the surface of each second angled plane allows tangential, sliding contact by the first angled plane of an advancement screw head. The one second angled plane of an advancement hole is contacted by the first angled plane of an advancement screw head for step compression or the other second angled plane of the advancement hole is contacted by the first angled plane of an advancement screw head for step distraction of the first bone segment relative to the second bone segment by advancement of the plate upon tightening one of the advancement screws into holes tapped into the second bone segment.

As with the system and the plate, the method can be performed by omitting the longitudinal slot and anchoring the plate by means other than anchoring screws. Additionally, it should be understood that the second angled planes of the advancement holes may be angled differently and/or differently spaced apart for effecting compression and distraction over different distances.

In one particular preferred embodiment of the aforementioned invention, the first angled plane of the advancement screw or other advancement mechanism may be achieved by use of a screw head which incorporates an inclined undersurface (for example: conical, spherical, oval or aspherical), herein referred to as advancement screws. In such an embodiment, the advancement feature of the plate body may include one or more corresponding countersunk holes, herein referred to as advancement holes, having a surface that define one or more second angled planes that allow tangential, sliding contact by the first angled plane of the advancement screw and forms a seat for receiving the head of the fully seated advancement screw.

In accordance with another feature of the invention, a plurality of advancement screws are sequentially tightened and loosened along the slot for continuing the step compression/distraction of the first bone segment relative to the second bone segment.

In accordance with one particular embodiment of the invention, one of the advancement screws may be tightened into the second bone segment to remain as an anchoring screw after a desired amount of compression/distraction is achieved.

In accordance with another embodiment of the invention, at least one additional anchoring screw is tightened through an additional anchoring hole in the plate in the vicinity of the advancement holes and remains in the second bone segment as an anchoring screw.

In accordance with an additional embodiment of the invention, an anchoring screw may be tightened through a multi-angle screw hole in the plate body and through the second bone segment into the first bone segment after a desired amount of compression has been achieved. This is particularly useful in an oblique osteotomy where the screw placed through the multi-angle screw hole traverses both bone segments and aids in stabilization of the bone segments.

In accordance with yet another embodiment of the invention a drill guide may be used to drill eccentrical tap holes for the advancement screws. This drill guide can be used for compression or distraction osteotomy, but is preferably used in distraction osteotomy, where the surgeon may make one free handed cut of the bone to be distracted without the use of a saw guide.

In another embodiment of the invention, there is provided a drill guide for a step compression/distraction osteotomy plate comprising a head having at least one drill guide hole formed therethrough for guiding a drill bit through the osteotomy plate and into a bone segment. In the present embodiment, at least one locating protrusion projects from the drill guide head for engaging an advancement hole in the osteotomy plate.

In accordance with another embodiment of the invention, there is provided a drill guide wherein at least one drill guide hole is off-center in the head for eccentrically drilling tap holes for bone advancement screws, or wherein the at least one drill guide hole is two or three drill guide holes, and the at least one locating protrusion is two or three locating protrusions each for engaging in a separate advancement hole in the osteotomy plate.

In accordance with another embodiment of the invention, a handle is connected to the head of the drill guide.

In accordance with a further embodiment of the invention, a directional indicator may be disposed on the head of the drill guide.

In accordance with yet another embodiment of the invention, a saw guide may be used to assist in cutting the bone and in drilling eccentrical tap holes for receiving the advancement screws. This saw guide may be used for compression or distraction osteotomy, but is preferably used for compression osteotomy, where the surgeon needs to make two substantially parallel cuts to the bone to be distracted. The substantially parallel cuts may be substantially perpendicular to the longitudinal axis of the bone to be distracted or they may be oblique to such axis.

In accordance with another embodiment of an osteotomy system of the present invention, there is provided a saw guide for a step compression/distraction osteotomy plate, wherein the saw guide comprises an elongated body with a distal end portion, a proximal end portion, a center portion defining a first cutting plane, an adjustable plate defining a second cutting plane substantially parallel to the first cutting plane and an adjustment screw that threadably engages a threaded hole in the adjustable plate, while freely rotating within non-threaded holes provided at each end of a center portion of the saw guide. In the instant embodiment, turning a knob in the adjustment screw displaces the adjustable plate within the center portion of the saw guide along the longitudinal axis of the elongated body of the saw guide in such a way that the separation between the first cutting plane and the second cutting plane can be adjusted by the surgeon to the desired length of bone to be cut. If desired, a marking scale may be provided in the central body portion to indicate the separation between the first cutting plane and the second cutting plane.

In accordance with another embodiment of the invention, the saw guide may be provided such that the first cutting plane in the center portion of the saw guide and the second cutting plane in the adjustable plate are substantially perpendicular to the longitudinal axis of the saw guide. Alternatively, or in addition to, the saw guide may be provided such that the first cutting plane in the center portion of the saw guide and the second cutting plane in the adjustable plate are oblique to the longitudinal axis of the saw guide. The angle formed between both cutting planes and the longitudinal axis of the saw guide may be any angle in the range of 30 degrees to 90 degrees (i.e., perpendicular) but, preferably, 45 degrees.

In accordance with yet another embodiment of the invention, a saw guide is provided which includes a slotted keyhole at each of the distal end portion and the proximal end portion of the saw guide body. As used herein, the term “slotted keyhole” denotes an elongated hole or slot, wherein the opening at a first end is dimensioned to be larger than the opening at the second, opposite end, such that the head of a screw may freely pass through the first end opening, but not through the second end opening. The hole or slot is dimensioned to permit the screw's shaft to move freely from one end of the slot to the opposite end.

In accordance with a further embodiment of the invention, there is provided a saw guide wherein the distal end portion of the saw guide includes at least one hole, in addition to the slotted keyhole, for receiving an anchoring screw. The saw guide of the present embodiment may additionally include, at its proximal end portion, at least two further holes serving as drill guides for eccentrically drilling tap holes in the bone for receiving advancement screws.

The osteotomy system of one particular embodiment of the present invention utilizes a combination step compression/distraction osteotomy plate including several advancement holes that may partially merge onto each other, in series, along a longitudinal slot on the plate. An osteotomy, creating two bone segments, may be performed by the surgeon free handed or, alternatively, with the aid of a saw guide. Tap holes for the advancement bone screws are drilled eccentrically using a drill guide or the saw guide. After fixing one end of the plate to a first bone segment an incremental advancement is obtained as the advancement screws are tightened and loosened sequentially into a second bone segment. In this manner, it is possible to advance the plate on the bone a large distance, compressing or distracting the bone segments. One of these advancement screws can remain as an anchoring screw after the bone translation is finished. The present system and method of allowing compression and/or distraction of an osteotomy facilitates the use of a smaller incision and reduces the number of procedural steps, time, and instruments that the procedure requires.

With the objects of the invention in view, there is provided a further embodiment of a step compression/distraction osteotomy system, comprising at least one wedge having a surface defining a first angled plane, and a plate having a distal end, a proximal end and first and second plate body portions sliding on one another. One of the plate body portions has at least one inclined surface facing the distal end and defining one second angled plane and at least one inclined surface facing the proximal end and defining another second angled plane, such second angled planes allowing tangential, sliding contact by the first angled plane. At least one advancement screw is provided for tightening the first angled plane against the one second angled plane for compression or against the other second angled plane for distraction of a bone segment attached to one of the plate body portions relative to another bone segment attached to the other of the plate body portions.

In accordance with another embodiment of the invention, at least two wedges are provided, each having a surface defining a first angled plane, for engaging a plurality of second angled planes on the osteotomy plate.

In accordance with a further embodiment of the invention, the first and second plate body portions have extensions sliding on one another and bases attached to the bone segments, and the second angled planes are on one of the extensions.

In accordance with an added feature of the invention, the one second angled plane and the other second angled plane can be disposed at different angles for compression and distraction by different amounts.

In accordance with an additional feature of the invention, the one plate body portion is T-shaped and the at least one advancement screw is two advancement screws each disposed on a respective side of a leg of the T-shaped plate body portion. The advancement screws can be screwed into pre-drilled, tapped holes in the other plate body or into the bone.

In accordance with yet another feature of the invention, the one plate body portion has two legs defining a slot therebetween through which the at least one advancement screw passes. The advancement screws can be screwed into pre-drilled, tapped holes in the other plate body or into the bone.

Other features which are considered as characteristic for the invention are set forth in the appended claims.

Although the invention is illustrated and described herein as embodied in an osteotomy system, plate, method, drill guide and saw guide, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWINGS

FIG. 1 is a diagrammatic, top-plan view of a step compression/distraction osteotomy plate according to a first embodiment of the invention;

FIG. 2 is a side-elevational view of the plate according to FIG. 1;

FIG. 3 is an enlarged, fragmentary, longitudinal-sectional view of the plate, which is taken along a line III-III of FIG. 1, in the direction of the arrows;

FIG. 4 is an end-elevational view of the plate according to FIG. 1;

FIG. 5 is a top-perspective view of the plate according to FIG. 1;

FIG. 6 is a bottom-perspective view of the plate according to FIG. 1;

FIG. 6A is a detailed, enlarged plan view of the distal end of the plate according to FIG. 1;

FIG. 6B is a detailed, enlarged plan view of the proximal end of the plate according to FIG. 1;

FIG. 7 is a top-plan view of the plate screwed to bone segments to be compressed;

FIG. 8 is a side-elevational view of the illustration according to FIG. 7;

FIG. 9 is an enlarged, fragmentary, longitudinal-sectional view of the plate, bone and screws, which is taken along a line IX-IX of FIG. 7, in the direction of the arrows;

FIG. 10 is a perspective view of the plate screwed to a bone to be compressed and a single-hole drill guide;

FIG. 11 is a perspective view of the single-hole drill guide showing the bottom thereof;

FIG. 12 is a fragmentary, enlarged, perspective view of a portion XII of the single-hole drill guide of FIG. 11;

FIG. 13 is a perspective view of the plate screwed to a bone to be compressed and a multiple-hole drill guide fitted to the plate;

FIG. 14 is a fragmentary, perspective view of the plate screwed to a bone after compression and a portion of the multiple-hole drill guide after removal;

FIGS. 15A-15H are perspective views of the plate, bone and screws illustrating a compression sequence;

FIG. 16 is a view similar to FIG. 14, additionally showing the screws within the plate and bone and internal channels of the drill guide, in broken lines;

FIG. 17 is a perspective view of the plate screwed to a bone after compression along an oblique cut;

FIG. 18 is a fragmentary, enlarged, perspective view of a portion XVIII of FIG. 17, additionally showing the screws within the plate and the bone;

FIGS. 19, 20 and 21 are two perspective views and a side-elevational view of a second embodiment of the step compression/distraction osteotomy plate according to the invention, screwed to a bone to be compressed;

FIG. 22 is an oblique plan view of the saw guide according to the invention;

FIG. 23 is an oblique exploded view of the saw guide of FIG. 22;

FIG. 24 is an oblique view of a saw guide and the bone segments after an oblique osteotomy has been performed;

FIG. 25 is a side elevational view of the saw guide of FIG. 22 screwed to the bone after an oblique osteotomy has been performed;

FIG. 26 is a side elevational view of the saw guide of FIG. 22 after it has been shifted distally, immediately prior to removal;

FIG. 27 is a side elevational view of the step compression/distraction plate according to FIG. 1 immediately after placement in substitution of the saw guide.

FIG. 28 is a side elevational view of the step compression/distraction plate of FIG. 27 that has been shifted proximally, prior to initiating the step advancement sequence.

### DETAILED DESCRIPTION OF THE INVENTION

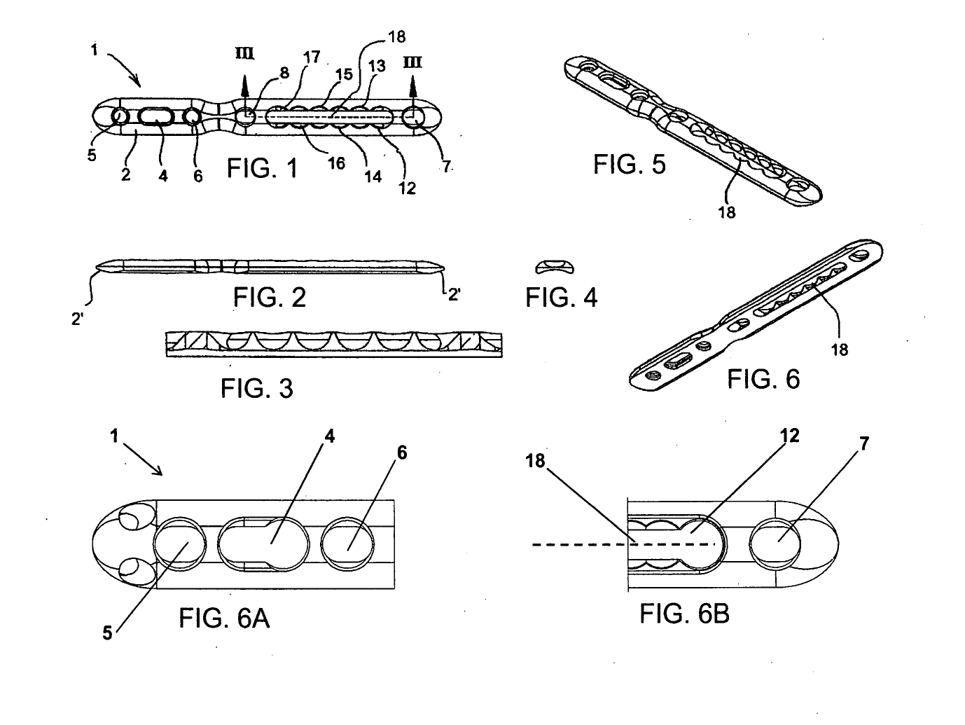

Referring now to the figures of the drawings in detail and first, particularly, to FIGS. 1-6B thereof, there is seen a step compression/distraction osteotomy plate 1 having a plate body 2 with a distal end 2′, a proximal end 2″, a slotted keyhole 4, anchoring screw holes 5, 6, 7, a multi-angle screw hole 8 and first through sixth advancement/distraction screw holes 12-17, formed therein, according to a first embodiment of the invention. Note that, although six advancement/distraction screw holes are illustrated in connection with the present embodiment, this is not meant to be limiting, as fewer or more advancement/distraction screw holes may be used will still keeping with the principles of the present invention. As is best seen in FIG. 5 and FIGS. 6 and 6B although the screw holes 12-17 have discrete curved surfaces, they are also interconnected and merge into one another along a continuous slot 18, permitting screws to be partially screwed-in between the holes and also permitting the plate to slide past the partially screwed-in screws. Additionally, and as best seen in FIGS. 6A and 6B, hole 4 and hole 12 are “slotted keyholes”, which herein denotes a hole that permits installation of the plate over the head of a screw that, for example, may previously have been positioned in the bone for alignment. The installation of a combination advancement/distraction plate, in accordance with the instant invention, will be further described herebelow in connection with a description of the novel saw guide useful with the osteotomy system of the present invention.

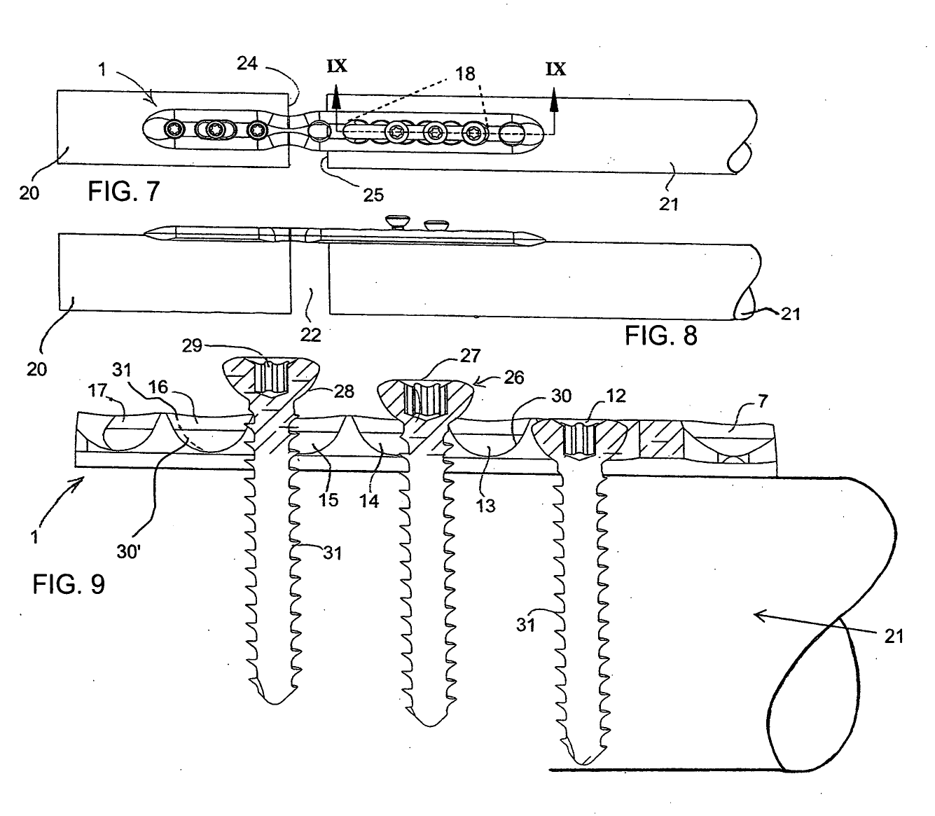

FIGS. 7-8 illustrate the position of the plate 1 on portions 20, 21 of a bone, which has been cut to remove a section of the bone defining a gap 22. The bone segments 20, 21 have respective surfaces 24, 25 to be compressed or placed into contact with one another. It may also be seen in FIG. 9, that a series of advancement screws 26 have been screwed through or between several of the advancement screw holes 12-17 and into bone segment 21. The screws have heads 27 with curved surfaces 28 and sockets 29 and screw threads 31. The sockets 29 have polygonal, for example hexagonal, hexalobular or multilobular, surfaces for receiving a driver for tightening and loosening the screws. The screw holes have similar countersunk curved surfaces 30 forming seats for the screws. However, the angle of the surfaces 30 may be varied from hole to hole and within one hole as seen by the surface 30′ shown dotted in hole 16. The compression/distraction screws and holes will be referred to below as advancement screws and advancement holes for simplicity.

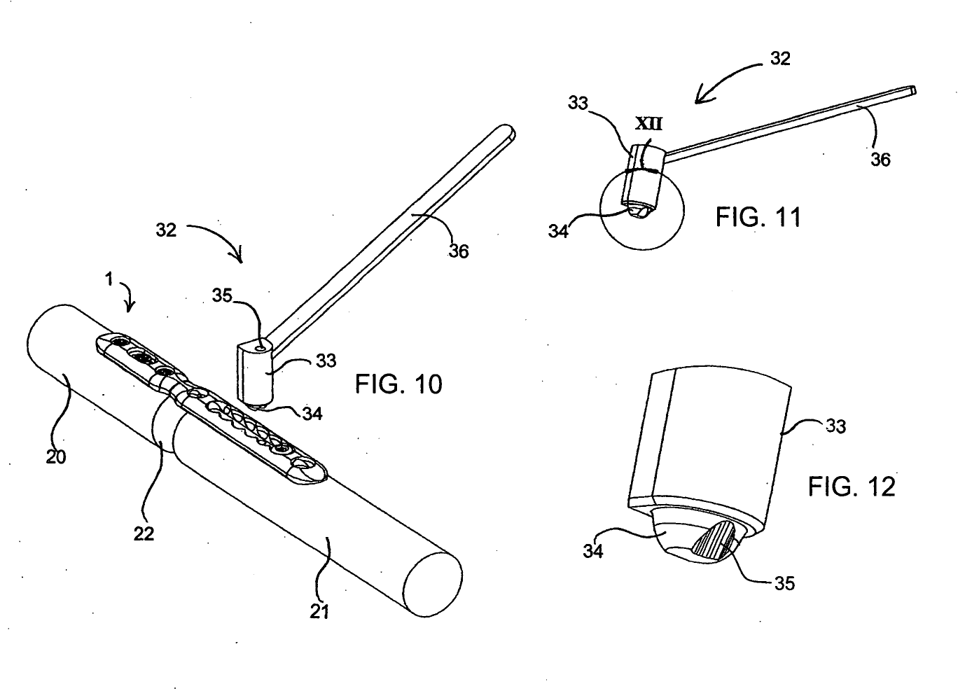

FIG. 10 shows the plate 1 screwed onto the bone segments 20, 21 having the gap 22. The figure also shows a single-hole drill guide 32 having a head 33 with a locating protrusion 34 and an eccentric drill guide hole 35, as well as a handle 36. The locating protrusion 34, which is used to position the head 33 above the step plate for drilling, is best seen in FIGS. 11 and 12. Drill guide 32 facilitates precise drilling of eccentric tap holes for accurate placement of the advancement screws into their initial positions.

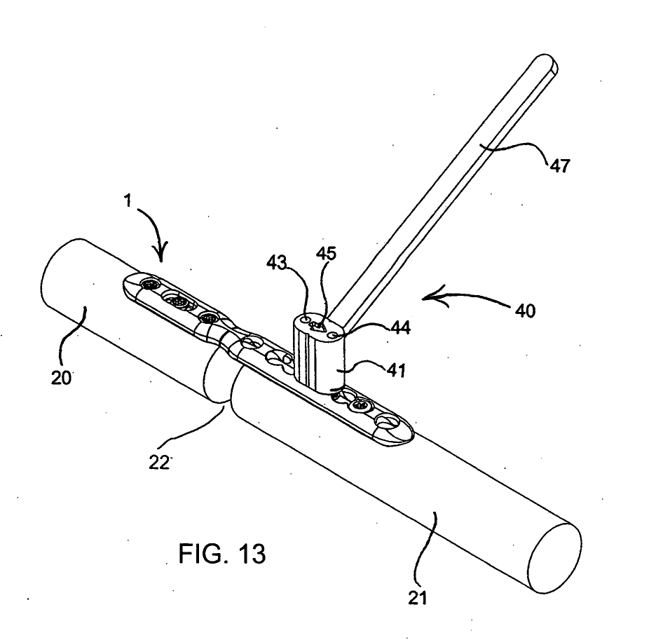

FIG. 13 similarly shows the plate 1 screwed onto the bone segments 20, 21 showing gap 22 before compression. However, a multiple-hole drill guide 40 is shown in FIG. 13. The drill guide 40 has a head 41 with two eccentrical drill guide holes 43, 44, between which a directional indicator 45 is disposed. A handle 47 for the drill guide 40 is also shown.

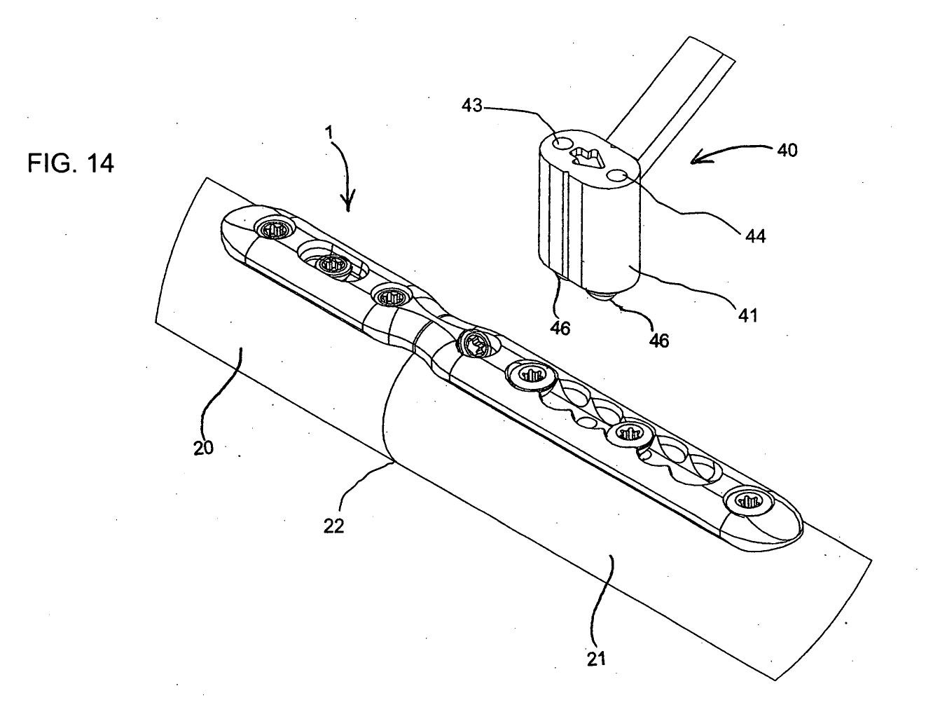

FIG. 14 illustrates the plate 1 screwed to the bone segments 20, 21, after compression, in which the gap 22 has been closed and the multiple-hole drill guide 40 with eccentrical guide holes 43, 44 has been removed. It may be seen from FIG. 14 that the head 41 has two locating protrusions 46 for positioning on the step plate.

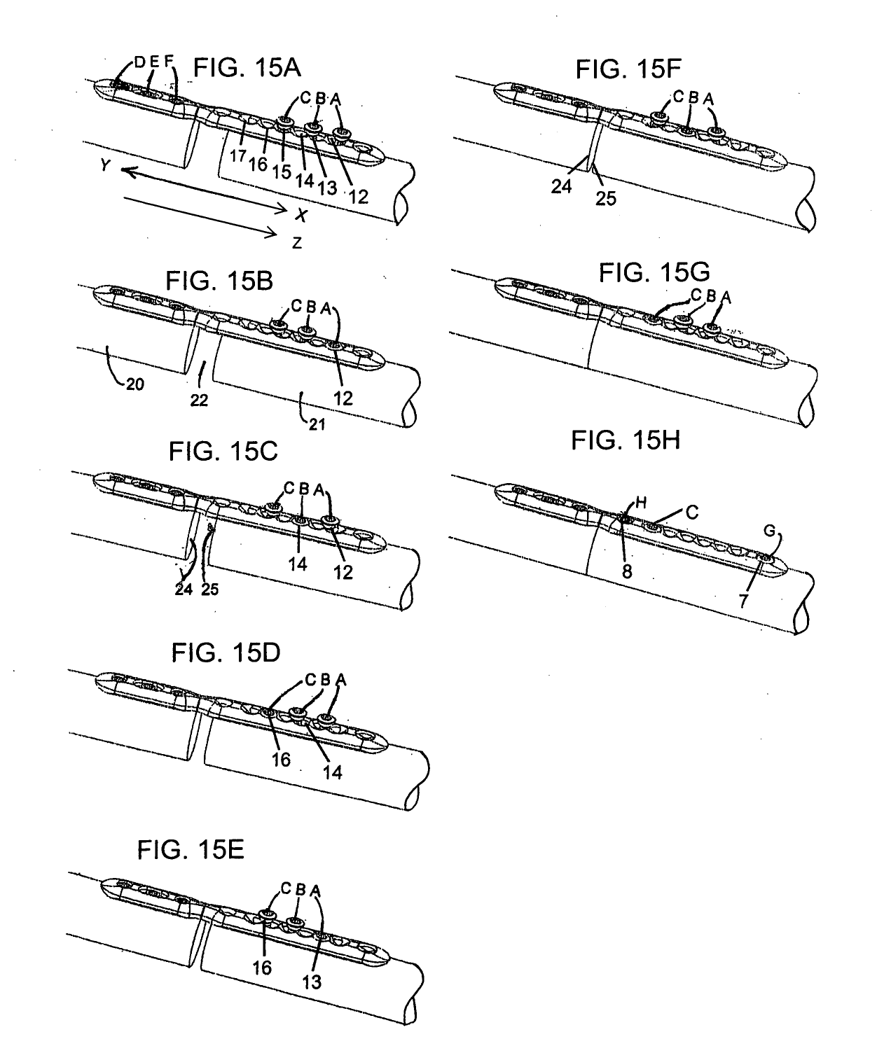

FIGS. 15A to 15H illustrate steps in a sequence of compressing the bone segments 20, 21 to bring the surfaces 24, 25 thereof into contact with one another and close the gap 22.

As mentioned above, the step plate functions by alternating advancement of a first angled plane in the screw head onto a corresponding second angled plane in the plate. Other second angled planes are arrayed to permit a sequence of advancements. In the preferred embodiment, as is illustrated in FIG. 9, the first angled planes are formed by the undersurfaces 28 of the screw heads 27, and the corresponding second angled planes are formed by the countersunk surfaces 30 of the holes 12-17 in the body of plate 1. The position of the screws 26 within the slot 18 in plate 1 in FIG. 7, insure that the plate can only advance along the axis of the slot 18.

However, according to the invention, the distance between the holes 12-17 may be varied and the angle of the surfaces 30 may be varied from hole to hole and within one hole as indicated dotted in 30′. In this way, different locations for the screws as well as different advancement distances provided by the angle of the surfaces 30 or 30′ may be chosen. Additionally, since portions of the surfaces 30 or 30′ of each hole face both the distal 2′ and proximal 2″ ends of the plate 1, each hole 12-17 can be used for compression and/or distraction of the bone segments through the use of one multi-function plate. These features give the surgeon a choice of advancement distance, advancement direction and screw location in one single plate. If needed, a screw can also be used for distraction of part of a distance advanced by compression, or vice-versa. Furthermore, the provision of the continuous slot 18 makes movement of the plate past a screw possible without removing the screw completely from the tap hole in the bone, which is advantageous in reducing surgical procedure times.

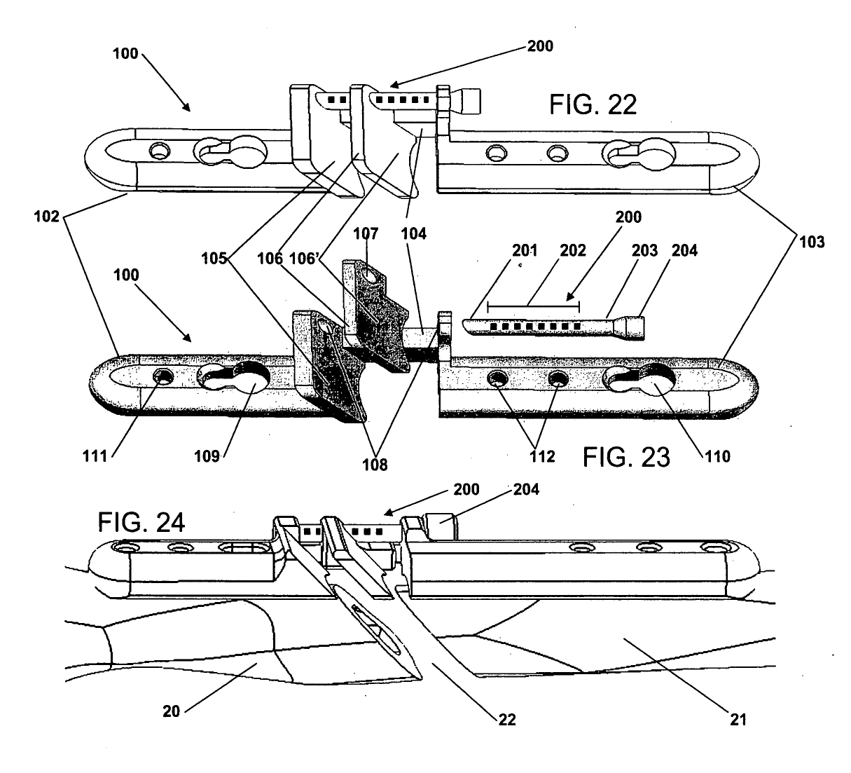

In FIGS. 15A-15H, several screws 26 (as seen in FIG. 9) will be referred to by reference symbols A-H in order to clearly identify the screws as a sequence of compression is described below. A proximal direction X and a distal direction Y of the plate will also be mentioned below. In each of FIGS. 15A-15H, first, second and third screws A, B, C are tightened and loosened in holes that have been drilled in the bone segment 21 with the aid of the drill guides 32 or 40 (FIGS. 10-14) or with the aid of a saw guide (100 of FIGS. 22-26), while the anchoring screws D, E, F pass through the respective holes 5, 4, 6 in the plate 1 into holes drilled in the bone segment 20, where they remain tightly in place.

The plate 1, along with the bone segment 20, is advanced in a bone movement direction Z by alternately securing the plate to the bone segment 21 with the first advancement screw A, the second advancement screw B and the third advancement screw C. The locations of the screws B and C, relative to the first screw A, and relative to the countersunk surfaces 30 of the holes 12-17 in the plate 1, are critical to permit advancement, which is why the drill guides 32, 40 (FIGS. 10 and 14) or, alternatively, the saw guide 100 (FIGS. 22-26) are used to properly position the tap holes for positioning the advancement screws eccentrically relative to 12-17.

In FIG. 15A, which shows the position of the plate prior to compression, the screws D, E, F are tightened in place in holes predrilled in the bone segment 20. Although the screws A, B, C have been positioned into eccentric tap holes predrilled in the bone segment 21 using the drill guide or the saw guide, none of them have been tightened. The plate 1 is affixed to the bone segment 21 by the first screw A, while the second and third screws B and C are partially screwed into predrilled tap holes, so that advancement can be achieved by sequential and alternate tightening and then loosening of each screw. For example, the screw A and the tap hole into which it is screwed may be placed 2 mm distally of the countersunk surface 30 of the first advancement screw hole 12. The screw B and the tap hole into which it is screwed may be positioned 2 mm distally of the countersunk surface 30 of the third advancement screw hole 14 and the screw C and the tap hole into which it is screwed may be positioned 2 mm proximally of the countersunk surface 30 of the fourth advancement screw hole 15.

According to FIG. 15B, the screw A is first tightened until it contacts the countersunk surface 30 of the first advancement screw hole 12. Continued tightening causes the plate to shift proximally (i.e., in direction X of FIG. 15A) by 1 mm.

According to FIG. 15C, once the screw A is fully seated in the countersunk surface 30 of the first advancement screw hole 12, the screw B is tightened until it contacts the countersunk surface 30 of the third advancement screw hole 14. This contact will secure the plate from moving distally. The screw A is then loosened until its head completely clears the plate, at which time the screw B is fully tightened, shifting the plate proximally an additional 1 mm.

According to FIG. 15D, once the screw B is fully seated in the countersunk surface 30 of the third advancement screw hole 14, the screw C is tightened until it contacts the countersunk surface 30 of the fifth advancement screw hole 16. This contact will secure the plate from moving distally. The screw B is then loosened until its head completely clears the plate, at which time screw C is fully tightened, shifting the plate proximally an additional 1 mm.

According to FIG. 15E, once the screw C is fully seated in the countersunk surface 30 of the fifth advancement screw hole 16, the screw A is tightened until it contacts the countersunk surface 30 of the second advancement screw hole 13. This contact will secure the plate from moving distally. The screw C is then loosened until its head completely clears the plate at which time screw A is fully tightened shifting the plate proximally another 1 mm.

According to FIG. 15F, the screw A is then loosened until its head completely clears the plate, at which time the screw B is fully tightened, shifting the plate proximally an additional 1 mm.

According to FIG. 15G, this sequence is continued by loosening the screw B and tightening the screw C, until sufficient compression of the surfaces 24, 25 is reached, or until the end of the slotted region of the plate is encountered. The total desired advancement may be, for instance 5-15 mm. Note that the total advancement performed can be greater than or less than the range of 5-15 mm.

Finally, according to FIG. 15H, the screws G and H are tightened through the holes 7 and 8 and predrilled holes in the bone segment 21 as anchoring screws to maintain the compression along with the screw C which is left in place, while removing the screws A and B. The bone will grow together to close the tap holes of screws A and B which will no longer be needed.

It is understood, that the sequence of steps described above may be carried out in reverse to perform distraction, that is, separation of the bone segments. It is also understood, as mentioned above and seen in FIG. 9, that the screws 26 and countersinks 30 may have different angled planes 30′ to provide different advancement and distraction. It is additionally understood that more or less than three screws 26 may be used and that the distance between the screw holes 12-17 may be varied. All of these variations can be used individually or in any combination to vary the translation of the plate and bone segment.

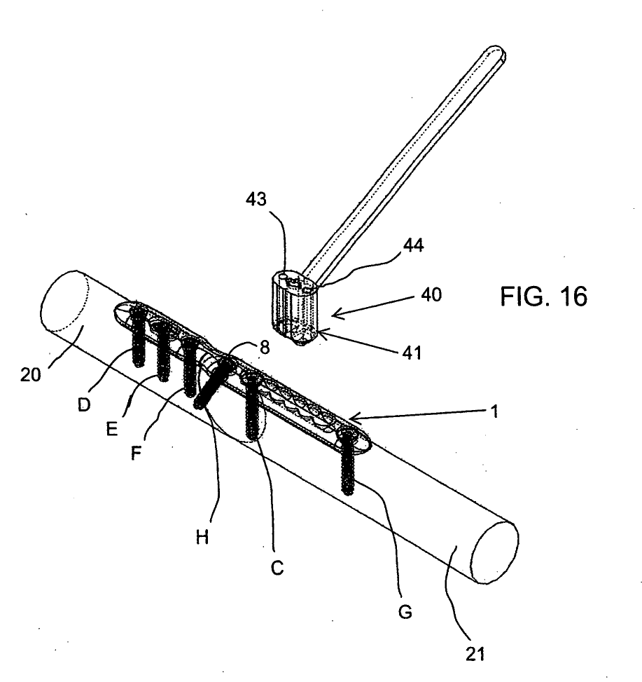

FIG. 16 shows the plate 1 secured on the bone segments 20, 21 after completing the bone advancement, as in FIG. 15H. The screws passing through the plate into the bone segments can also be seen. In particular, it is shown that the screw H has been screwed-in obliquely in the multi-angle screw hole 8, so that it is screwed from the bone segment 21 into the bone segment 20. It is additionally shown how the drill guide holes 43, 44 in the head 41 of the multiple-hole drill guide 40 extend through the head to allow drilling of the eccentric tap holes.

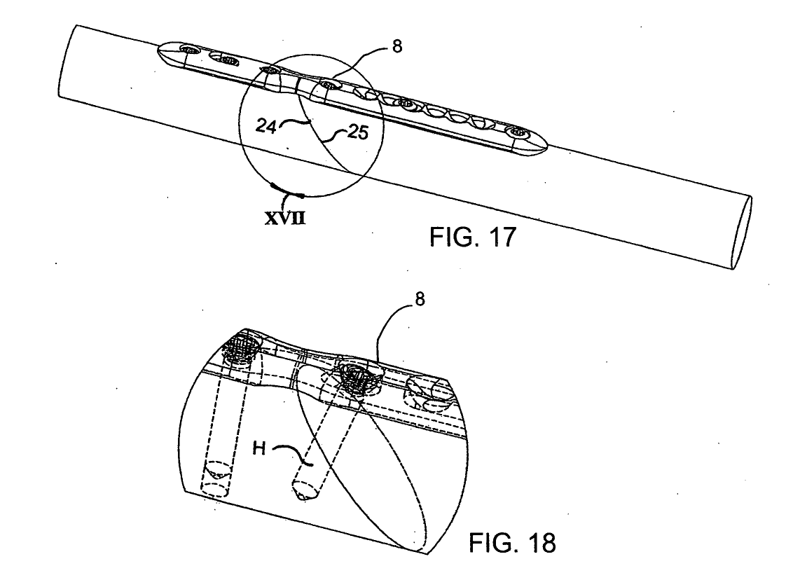

FIGS. 17 and 18 similarly show the screw H passing through the multi-angle screw hole 8, into the bone segment 21 and into the bone segment 20. However, the surfaces 24 and 25 have been cut at an oblique angle, for example, using the saw guide 100 of FIGS. 22-26. The saw guide 100 of FIGS. 22-26 is advantageously adapted to cut the bone at a oblique angle. One or more of the holes 12-17 may also be multi-angle screw holes.

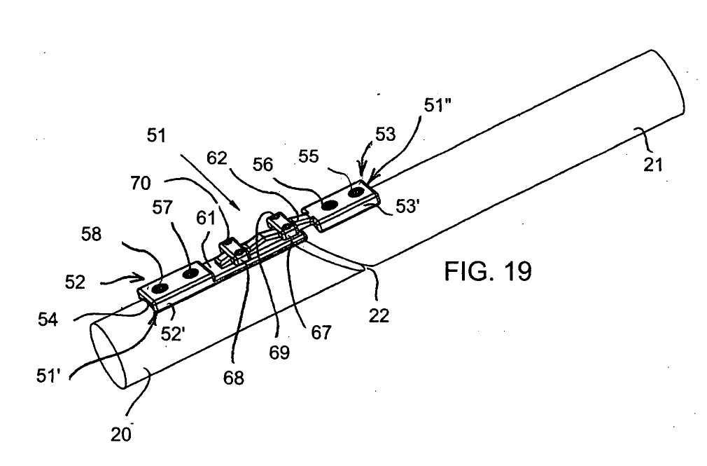

FIGS. 19 and 20-21 illustrate second and third embodiments of a step compression/distraction osteotomy plate 51 according to the invention, which is screwed to the bone segments 20, 21 and has a distal end 51′ and a proximal end 51″. The plate 51 includes a first plate body portion 52 and a second plate body portion 53. Both plate body portions 52, 53 have concave lower surfaces 54 resting on the bone segments.

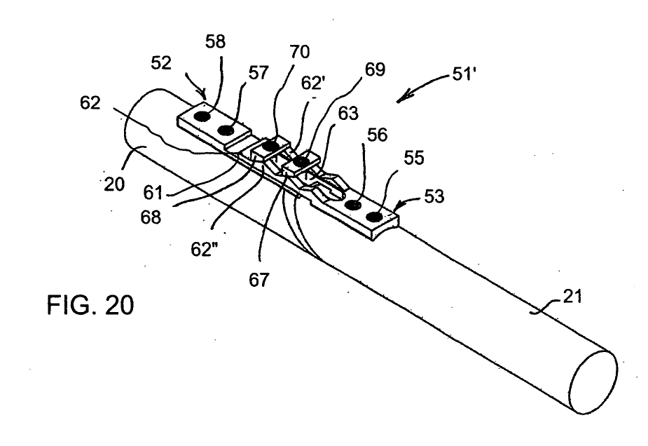

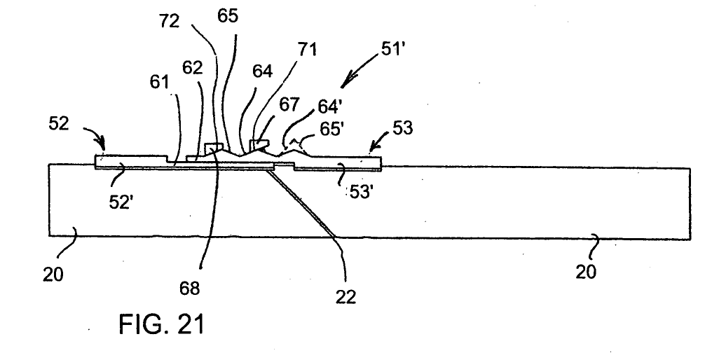

The first plate body portion 52 has anchoring screw holes 57, 58 and the second plate body portion 53 has anchoring screw holes 55, 56. The first and second plate body portions 52, 53 have respective first and second bases 52′, 53′ and first and second extensions 61, 62, as is best seen in FIGS. 19 and 21. The first extension 61 has upper and lower planar surfaces and the second extension 62 has a lower planar surface sliding on the upper planar surface of the first extension 61. It is also noted that the second extension 62 in FIG. 19 is narrower than the base 53′ of the second plate body portion 53, giving the second plate body portion 53 a T-shaped profile as seen in a plan view. However, in FIG. 20, the second extension 62 has the same width as the remainder of the second plate body portion 53. In addition, the second extension 62 in FIG. 19 is a single leg, whereas the second extension 62 in FIG. 20 is bifurcated into two legs 62′, 62″ defining a slot 63 therebetween.

In FIGS. 19, 20 and 21, the upper surface of the second extension 62 has a sawtooth profile with crests, valleys and inclined surfaces therebetween.

The inclined surfaces include second angled planes 64 for distraction and second angled planes 65 for compression, of the bone segments 20, 21 in FIG. 21. It may be seen that wedges 67, 68 are disposed on the second angled planes 64. The wedges 67, 68 have respective holes 69, 70 formed therein. The wedges 67, 68 each have two respective holes 69, 70 in FIG. 19 disposed laterally outside of the second extension 62. The wedges 67, 68 each have one respective hole 69, 70 in FIG. 20 disposed above the slot 63. The holes 69, 70 receive screws for tightening the wedges 67, 68 against the second angled planes 64. The wedges 67, 68 have respective oblique surfaces 71, 72 acting as first angled planes contacting the second angled planes 64 or 65 wherein the angles of the first and second angled planes are substantially complementary.

In order to open or close the gap 22 between the bone segments 20, 21 (FIG. 19) anchoring screws are screwed through the holes 55, 56 and 57, 58 in the respective plate body portions 53 and 52. Then, screws are tightened into pre-tapped holes in first extension 61 through the holes 69, 70 in the wedges 67, 68, so that the wedges are pressed against and slide down along whichever second angled planes, 64 or 65, they are disposed above.

In the position shown in FIG. 21, the first angled planes 71, 72 of the wedges 67, 68 are disposed on the second angled planes 64, so that when they are tightened downward with screws, they will push the second plate body portion 53 and therefore the bone segment 20 to the right in FIG. 21, causing distraction and widening of the gap 22. After the step distraction, for example by a distance of 1 mm, the screws may be removed, the wedges may be repositioned at the top of the second angled planes 64 and the screws may be tightened once again to move the second plate body portion 53 and the bone segment 20 to the right by an additional 1 mm. This procedure is repeated until the desired amount of distraction has been achieved.

If step compression is desired, the wedges 67, 68 are reversed and the first angled planes 71, 72 of the wedges are placed at the top of the second angled planes 65, so that tightening of the screws will move the second plate body portion 53 and the bone segment 20 to the left. Of course, the wedges and planes may be dimensioned to provide compression/distraction of more or less than 1 mm. In addition, the angles of the first angled planes 71, 72 of the wedges and the angles of the second angled planes 64, 65 may be varied as seen by dotted lines 64′ and 65′ so as to vary the compression and distraction distances. It is also possible to provide a surface 64 and a surface 65′ on two sides of a single crest or a surface 64′ and a surface 65 on two sides of a single crest, so that different compression and distraction distances are covered by using either side of a single crest.

In a manner similar to the embodiment of FIGS. 1-18, in this embodiment as well, the distance between the crests, valleys and inclined surfaces may be varied and the angle of the surfaces may be varied from one to the next. In this way, different locations for the screws as well as different advancement distances provided by the angle of the surfaces may be chosen. The fact that the surfaces face toward both the distal and proximal ends 51′, 51″ of the plate 51 (FIG. 19) and the fact that each set of crests, valleys and inclined surfaces can be used for compression and/or distraction of the bone segments through the use of one multi-function plate, again give the surgeon a choice of advancement distance, advancement direction and screw location in one single plate. A screw can also be used for distraction of part of a distance previously advanced by compression, or vice versa. Furthermore, the provision of the continuous slot 63 makes movement of the plate past a screw possible without removing the screw completely from the plate, which is advantageous given space limitations at a surgical site. A screw may simply be backed-off, a wedge on the screw reversed and the plate slid past the screw so that the wedge can be placed on the other side of a crest, to change between compression and distraction.

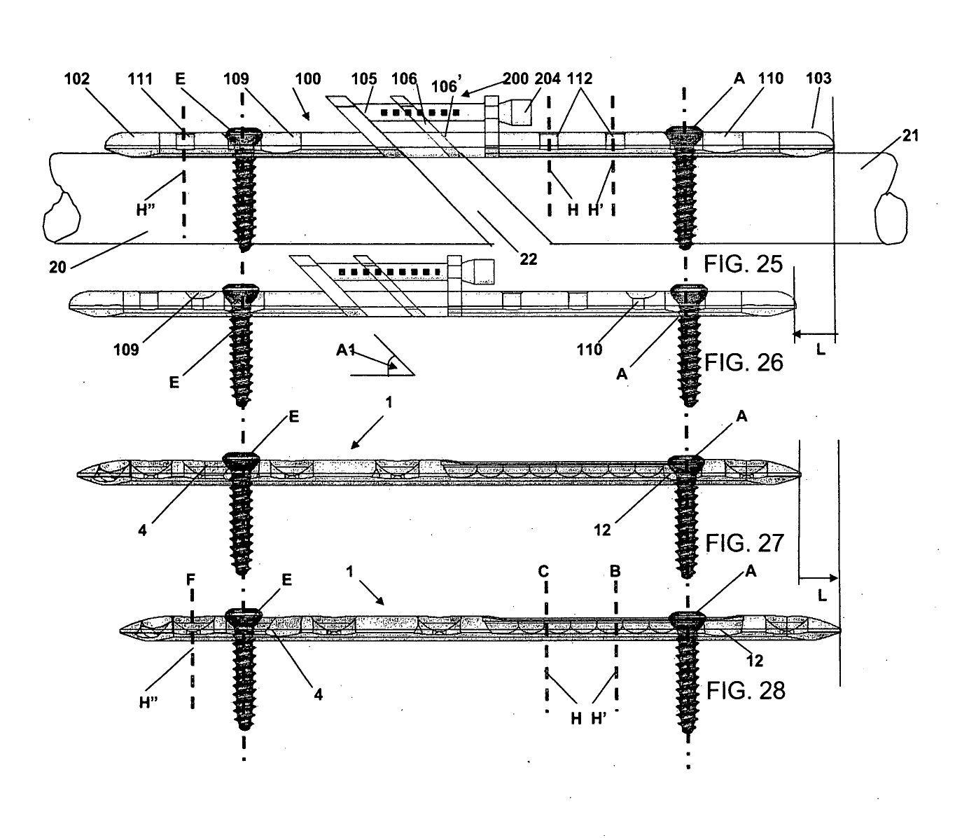

Referring now to FIGS. 22-24, there is one particularly preferred embodiment of a saw guide 100, which may be used with the osteotomy system of the instant invention. The saw guide 100 may be used as a guide during the performance of an osteotomy and, additionally, for the initial alignment of a step compression/distraction osteotomy plate, for example, the osteotomy plates 1, 51, 51′ of FIGS. 1, 19 and 20, just prior to step compression or distraction. Although the saw guide 100 may be used for compression or distraction osteotomy, the embodiment shown is more advantageously used for compression osteotomy, where the surgeon needs to make two substantially parallel cuts to the bone being compressed. The saw guide 100 is comprised of an elongated body having a distal end portion 102, a proximal end portion 103, a center portion 104. The center portion 104 of the saw guide 100 includes a fixed plate 105, which defines a first cutting plane, an adjustable plate 106, defining a second cutting plane 106′, substantially parallel to the first cutting plane 105. An adjustment screw 200 threadably engages the adjustable plate 106, through threaded hole 107. However, non-threaded holes 108, provided at each end of the center portion 104, are sized to allow the adjustment screw 200 to rotate freely in the non-threaded holes 108. To ensure this free rotation, the adjustment screw 200 has a non-threaded distal end 201 and a non-threaded proximal end 203, which pass through the non-threaded holes 108. Simultaneously, threads formed in the threaded central portion 202 of the adjustment screw 200 engage the threads of the adjustable plate threaded hole 107. By turning the adjustment screw knob 204 of the adjustment screw 200 clockwise or counterclockwise, the adjustable plate can be respectively displaced distally or proximally within the center portion 104, along the longitudinal axis of the elongated body of the saw guide 100, to permit the surgeon to adjust the separation between the first cutting plane 105 and the second cutting plane 106′ to the desired bone gap length (For example, 22 of FIGS. 24, 25) to be cut. If desired, the central portion 104 of the saw guide 100 can be provided with a marking scale (not shown), to assist the surgeon by indicating the separation between the first cutting plane 105 and the second cutting plane 106′.

Note that, in the preferred embodiment shown in FIGS. 24-26, the first cutting plane 105 and the second cutting plane 106′ are substantially oblique to the longitudinal axis of the saw guide 100. Although not shown in FIGS. 24-26, if desired, center portion 104 of the saw guide 100 may be provided, alternatively, or in addition thereto, with a first cutting plane 105 and a second cutting plane 106′ that are perpendicular to the longitudinal axis of the saw guide 100. Furthermore, as shown more particularly in FIG. 26, the saw guide 100 may be oriented such that the angle formed between each cutting plane 105, 106′ and the longitudinal axis of the saw guide defines an angle A1, which is preferably in the range of 30 degrees to 90 degrees (perpendicular) but, more preferably, A1 is 45 degrees (as shown in FIG. 26).

Additionally, as discussed elsewhere herein, the saw guide 100 of the instant embodiment is provided with slotted keyholes 109, 110, which may be provided in the distal end portion 102 and the proximal end portion 103 of the body of the saw guide 100. Each of the slotted keyholes 109, 110 defines an elongated hole or “slot”, wherein the width of the opening through the slot is greater at one end of the slot than at the other. In this way the slot is shaped as a “keyhole”, wherein the wider opening of the slot is dimensioned to permit the head of a screw to pass freely therethrough, while the smaller opening does not. In this way, the keyhole 109, 110 can accept the head of a screw therethrough at one end, but entrap the screw in the narrower opening of the slot. The slot, itself, is desirably dimensioned to permit the screw shaft to freely travel the length of the slot 109, 110.

As further shown in FIGS. 23 and 25, if desired, the distal end portion 102 of the saw guide 100 may be provided with at least one additional hole 111 to serve as a drill guide for drilling a tap hole (H″ of FIG. 25) in the bone. Such a tap hole H″ would, resultantly, be dimensioned to receive an anchoring screw. Similarly, if desired, the proximal end portion 103 of the saw guide 100 may be provided with additional hole(s), for example, holes 112 of FIG. 23, to serve as drill guides for eccentrically drilling tap holes (H, H′ of FIG. 25) in the bone for receiving advancement screws. Although two holes 112 are shown, it should be noted that more than two holes 112 can be provided, if desired.

Referring more particularly to FIGS. 25-28, there will now be described a method of using a saw guide 100 in accordance with one particular embodiment of the present invention. In particular the method for using the saw guide 100 can be used as a guide when performing an oblique compression osteotomy requiring making two cuts of a bone, drilling the required tap holes and installing step compression/distraction plate (such as, plates 1, 51 or 51′ of FIGS. 1, 19 and 20), prior to carrying out the compression. Although the method of using the saw guide 100 will be described in connection with the performance of an oblique compression osteotomy, it can be seen from the present descriptions that a distraction osteotomy can also be performed using the saw guide 100. In particular, it will be understood that a distraction osteotomy can be performed following the desired steps, however, wherein a single cut of a bone is preferably performed with a saw guide 100 having the cutting planes (105, 106′) oriented perpendicular to the longitudinal axis of the saw guide 100 (not shown).

Referring back to FIGS. 25-28, a saw guide 100 is attached to a bone using an anchoring screw (E of FIG. 15A) placed and tightened through the narrow portion of slotted keyhole 109 and an advancement screw (A of FIG. 15A) placed and tightened through the narrow portion of slotted keyhole 110. After the saw guide 100 has been secured in place (FIG. 25), the surgeon adjusts the position of adjustable plate 106 so that the distance between cutting plane 105 and cutting plane 106′ defines a distance that will produce an osteotomy resulting in the desired gap 22 between distal bone segment 20 and proximal bone segment 21. After setting the distance between the planes 105 and 106′, the surgeon makes two cuts in the bone by sequentially aligning a cutting saw (not shown) with the cutting planes 105 and 106′. The surgeon then removes the cut piece of bone, thereby creating the gap 22.

Referring more particularly to FIG. 26, after the desired gap (22 of FIG. 25) has been created, the screws A and E are loosened just enough to permit saw guide 100 to be displaced distally (i.e., in direction Y of FIG. 15A) by a distance L along the longitudinal axis of the slotted keyholes 109 and 110. Since the openings on the wide end of slotted keyholes 109 and 110 are dimensioned to be larger than the screw heads of screws E and A, the saw guide 100 can be removed by merely lifting it upwards, clearing the heads of the screws A and E. Screws E and A, however, are left attached to bone segments 20 and 21 in their slightly loosened positions.

Referring now to FIG. 27, after the removal of the saw guide 100 of FIG. 26, the combination step compression/distraction plate 1 (or, 51 or 51′ of FIGS. 19 and 20) can be positioned over the screws E and A, still in their slightly loosened positions. For example, a, step compression/distraction plate 1 (see also, FIGS. 1, 6A and 6B) can be positioned above the bone segments 20 and 21, such that the heads of the screws E and A are aligned the wider portions of the slotted keyholes 4 and 12, respectively. The step compression/distraction plate 1, 51, 51′ is lowered over and past the screw heads E and A until the plate contacts the bone, and is seated on bone segments 20 and 21.

Referring now to FIG. 28, the step compression/distraction plate 1, 51 or 51′ is displaced proximally (direction X of FIG. 15A) by a distance L in such a way that the shafts of anchoring screw E and advancement screw A are positioned at the distal ends of the narrow portions of slotted keyholes 4 and 12, respectively. Anchoring screw E is then tightened into bone segment 20, thereby fixing the plate 1, 51, 51′ to the bone segment 20 on its distal end 102. An additional anchoring screw F may be placed into tapped hole H″ and tightened, further fixing the plate 1, 51, 51′. In the present embodiment, the advancement screw A remains loose, while the advancement screws B and C (shown in FIG. 15A) are loosely screwed through the continuous slot (18 of FIGS. 5, 6 and 7) into tapped holes H′ and H, respectively.

Once the step described in connection with FIG. 28 is accomplished, the step compression sequence described above in connection with FIGS. 15A-15H can be executed by the surgeon.

1. A step compression/distraction osteotomy system, the system comprising:
anchoring screws each having screw threads and a screw head;
advancement screws each having screw threads and a screw head with a surface defining a first angled plane;
a plate having a plate body with a distal end and a proximal end;
said plate body having anchoring holes formed therein for receiving said anchoring screws to fasten said plate to a first bone segment; and
said plate body having a longitudinal slot formed therein and having a series of advancement holes formed therein and merging into each other along said longitudinal slot;
said advancement holes each having a countersunk surface forming a seat for receiving said advancement screws to be screwed into a second bone segment, said countersunk surfaces each defining one second angled plane facing said distal end and another second angled plane facing said proximal end;
said surface defining said first angled plane contacting said one second angled plane for step compression or said other second angled plane for step distraction of the first bone segment relative to the second bone segment by advancement of said plate upon one of said advancement screws being screwed into the second bone segment.

2. The system according to claim 1, wherein a plurality of said advancement screws are tightened and loosened sequentially along said slot for continuing said selective step compression or distraction of the first bone segment relative to the second bone segment.

3. The system according to claim 2, wherein one of said advancement screws remains tightened in the second bone segment as an anchoring screw after a desired amount of compression.

4. The system according to claim 3, wherein at least one additional screw is tightened through an additional hole in vicinity of said advancement holes and remains in the second bone segment as an anchoring screw.

5. The system according to claim 1, wherein said plate body also has a multi-angle screw hole formed therein for receiving a screw passing through the second bone segment into the first bone segment after the bone segments are positioned.

6. The system according to claim 1, wherein said one second angled plane and said other second angled plane are disposed at different angles within at least one of said countersunk surfaces for compression and distraction by different amounts.

7. A step compression/distraction osteotomy system, the system comprising:
advancement screws each having a screw head defining a first angled plane; and
a plate body having a distal end, a proximal end, and a series of advancement holes;
at least some of said advancement holes having countersunk surfaces defining one second angled plane facing said distal end and another second angled plane facing said proximal end;
said first angled plane contacting said one second angled plane for step compression or said other second angled plane for step distraction of a bone segment relative to another bone segment by advancement of said plate upon one of said advancement screws being screwed into the other bone segment.

8. The system according to claim 7, wherein said one second angled plane and said other second angled plane are disposed at different angles within at least one of said countersunk surfaces for compression and distraction by different amounts.

9. A step compression/distraction osteotomy plate comprising:
a plate body with a distal end and a proximal end;
said plate body having anchoring holes formed therein for receiving anchoring screws to fasten said plate to a first bone segment;
said plate body having a longitudinal slot formed therein and having a series of advancement holes formed therein and merging into each other along said longitudinal slot;
said advancement holes each having a countersunk surface forming a seat for receiving advancement screws having a screw head defining a first angled plane;
said countersunk surfaces each defining one second angled plane facing said distal end and another second angled plane facing said proximal end; and
said one second angled plane to be contacted by a first angled plane for step compression or said other second angled plane to be contacted by a first angled plane for step distraction of the first bone segment relative to a second bone segment by advancement of said plate upon an advancement screw being screwed into the second bone segment.

10. The plate according to claim 9, wherein a plurality of the advancement screws are tightened and loosened sequentially along said slot for continuing said step compression/distraction of the first bone segment relative to the second bone segment.

11. The plate according to claim 10, wherein one of the advancement screws remains tightened in the second bone segment as an anchoring screw after the bone segments are positioned.

12. The plate according to claim 11, wherein at least one additional screw is tightened through an additional hole in vicinity of said advancement holes and remains in the second bone segment as an anchoring screw.

13. The plate according to claim 9, wherein said plate body also has a multi-angle screw hole formed therein for receiving a screw passing through the second bone segment into the first bone segment after the bone segments are positioned.

14. The plate according to claim 9, wherein said one second angled plane and said other second angled plane are disposed at different angles within at least one of said countersunk surfaces for compression and distraction by different amounts.

15. A step compression/distraction osteotomy plate comprising:
a plate body having a distal end, a proximal end, and a series of advancement holes;
said advancement holes each having a countersunk surface forming a seat for receiving advancement screws having a screw head defining a first angled plane;
at least some of said countersunk surfaces defining one second angled plane facing said distal end and another second angled plane facing said proximal end;
said one second angled plane to be contacted by a first angled plane for step compression or said other second angled plane to be contacted by a first angled plane for step distraction of a first bone segment anchored to said plate body relative to a second bone segment by advancement of said plate upon an advancement screw being screwed into the second bone segment.

16. The plate according to claim 15, wherein said one second angled plane and said other second angled plane are disposed at different angles within at least one of said countersunk surfaces for compression and distraction by different amounts.

17. A step compression/distraction osteotomy method, the method comprising the following steps:
providing anchoring screws each having screw threads and a screw head;
providing advancement screws each having screw threads and a screw head with a surface defining a first angled plane;
providing a plate having a plate body with a distal end and a proximal end;
providing anchoring holes in the plate body for receiving the anchoring screws to fasten the plate to a first bone segment;
providing a longitudinal slot in the plate body;
providing a series of advancement holes in the plate body merging into each other along the longitudinal slot, the advancement holes each having a countersunk surface defining one second angled plane facing the distal end and another second angled plane facing the proximal end as a seat for receiving the advancement screws to be screwed into a second bone segment; and
contacting, with the first angled plane, the one second angled plane for step compression or the other second angled plane for step distraction of the first bone segment relative to the second bone segment by advancement of the plate upon tightening one of the advancement screws into the second bone segment.

18. The method according to claim 17, which further comprises sequentially tightening and loosening a plurality of the advancement screws along the slot for continuing the step compression/distraction of the first bone segment relative to the second bone segment.

19. The method according to claim 18, which further comprises leaving one of the advancement screws tightened in the second bone segment as an anchoring screw after the bone segments are positioned.

20. The method according to claim 19, which further comprises tightening at least one additional screw through an additional hole in vicinity of the advancement holes and leaving the at least one additional screw in the second bone segment as an anchoring screw.

21. The method according to claim 17, which further comprises tightening a screw through a multi-angle screw hole in the plate body and through the second bone segment into the first bone segment after the bone segments are positioned.

22. The method according to claim 17, which further comprises drilling eccentric tap holes for the advancement/distraction screws with a drill guide.

23. The method according to claim 17, which further comprises forming the one second angled plane and the other second angled plane at different angles within at least one of the countersunk surfaces for compression and distraction by different amounts.

24. A step compression/distraction osteotomy method, the method comprising the following steps:
providing advancement screws each having screw threads and a screw head with a surface defining a first angled plane;
providing a plate having a plate body with a distal end and a proximal end;
anchoring to a first bone segment;
providing a series of advancement holes in the plate body each having a countersunk surface defining one second angled plane facing the distal end and another second angled plane facing the proximal end as a seat for receiving the advancement screws to be screwed into a second bone segment; and
contacting, with the first angled plane, the one second angled plane for step compression or the other second angled plane for step distraction of the first bone segment relative to the second bone segment by advancement of the plate upon tightening one of the advancement screws into the second bone segment.

25. The method according to claim 24, which further comprises forming the one second angled plane and the other second angled plane at different angles within at least one of the countersunk surfaces for compression and distraction by different amounts.

26. A step compression/distraction osteotomy system, the system comprising:
at least one wedge having a surface defining a first angled plane;
a plate having a distal end, a proximal end, and first and second plate body portions sliding on one another, one of said plate body portions having at least one inclined surface facing said distal end and defining one second angled plane and at least one inclined surface facing said proximal end and defining another second angled plane; and
at least one advancement screw for tightening said first angled plane against said one second angled plane for compression or against said other second angled plane for distraction of a bone segment attached to one of said plate body portions relative to another bone segment attached to the other of said plate body portions.

27. The system according to claim 26, wherein said at least one wedge is at least two wedges each having a surface defining a first angled plane; and
said second angled plane is one of a plurality of second angled planes.

28. The system according to claim 27, wherein said first and second plate body portions have extensions sliding on one another and bases attached to the bone segments, and said second angled planes are on one of said extensions.

29. The system according to claim 26, wherein said one second angled plane and said other second angled plane are disposed at different angles for compression and distraction by different amounts.

30. The system according to claim 26, wherein said one plate body portion is T-shaped and said at least one advancement screw is two advancement screws each disposed on a respective side of a leg of said T-shaped plate body portion.

31. The system according to claim 26, wherein said one plate body portion has two legs defining a slot therebetween through which said at least one advancement screw passes.

32. A drill guide for an osteotomy plate, the drill guide comprising:
a head having at least one drill guide hole formed therethrough for guiding a drill bit through the osteotomy plate and into a bone segment; and
at least one locating protrusion projecting from said head for engaging a hole in the osteotomy plate.

33. The drill guide according to claim 32, wherein said at least one drill guide hole is off-center in said head for eccentrically drilling tap holes for bone advancement screws.

34. The drill guide according to claim 32, wherein said at least one drill guide hole is two drill guide holes, and said at least one locating protrusion is two locating protrusions each for engaging in a separate hole in the osteotomy plate.

35. The drill guide according to claim 32, which further comprises a handle connected to said head.

36. The drill guide according to claim 32, which further comprises a directional indicator on said head.

37. The drill guide according to claim 32, wherein said at least one drill guide hole is three drill guide holes, and said at least one locating protrusion is three locating protrusions each for engaging in a separate hole in the osteotomy plate.

38. The step compression/distraction osteotomy system of claim 1, wherein at least one anchoring hole is a keyhole.

39. The step compression/distraction osteotomy system of claim 38, wherein at least one advancement hole of said series is a keyhole.

40. The step compression/distraction osteotomy system of claim 1, wherein at least one advancement hole of said series is a keyhole.

41. The step compression/distraction osteotomy plate of claim 9, wherein at least one anchoring hole is a keyhole.

42. The step compression/distraction osteotomy plate of claim 9, wherein at least one advancement hole of said series is a keyhole.

43. The step compression/distraction osteotomy plate of claim 15, wherein at least one anchoring hole is a keyhole.

44. The step compression/distraction osteotomy plate of claim 15, wherein at least one advancement hole of said series is a keyhole.

45. The method of claim 22, wherein the eccentric tap holes are drilled using a saw guide.

46. The method of claim 17, wherein at least one anchoring screw is used to fasten a saw guide to the first bone segment, prior to fastening the plate to the first bone segment.

47. The method of claim 17, wherein at least one advancement screw is used to fasten a saw guide to the second bone segment, prior to fastening the plate to the first bone segment.

48. The method of claim 17, wherein at least one advancement screw is used to orient a saw guide relative to the second bone segment, prior to contacting the plate.

49. A saw guide for assisting in an osteotomy, the saw guide comprising:
a body portion including a proximal end portion, a distal end portion and a center portion;
the center portion including a first cutting plane and a second cutting plane, the position of said second cutting plane being adjustable relative to the first cutting plane;
the distal end portion including a first hole for receiving a screw to anchor the distal end portion of the saw guide to a first bone portion; and
the proximal end portion including a second hole for receiving a screw to anchor the proximal end portion of the saw guide to a second bone portion.

50. The saw guide of claim 49, wherein the first cutting plane and the second cutting plane are oriented parallel to each other and perpendicular to a longitudinal axis through the body of the cutting guide.

51. The saw guide of claim 49, wherein the first cutting plane and the second cutting plane are oriented parallel to each other and obliquely to a longitudinal axis through the body of the cutting guide.

52. The saw guide of claim 51, wherein the cutting planes are oriented at a 45° angle to the longitudinal axis through the body of the cutting guide.

53. The saw guide of claim 49, wherein the second cutting plane is defined by an adjustable plate that is movable along the longitudinal axis of the saw guide.

54. The saw guide of claim 53, wherein the adjustable plate includes a threaded hole for engaging the threads on the shaft of an adjustment screw.

55. The saw guide of claim 54, wherein the first cutting plane is defined by a fixed plate oriented parallel to the adjustable plate, said fixed plate including a non-threaded hole therethrough, a non-threaded portion of said adjustment screw passing through said non-threaded hole.

56. The saw guide of claim 55, wherein rotation of said adjustment screw moves said adjustable plate relative to said fixed plate.

57. The saw guide of claim 49, wherein said first hole comprises an elongated slot.

58. The saw guide of claim 57, wherein said elongated slot is a keyhole.

59. The saw guide of claim 57, wherein said second hole additionally comprises an elongated slot.

60. The saw guide of claim 59 wherein said first hole and said second hole are keyholes.

61. A method for using a saw guide to remove a desired length of bone for an osteotomy, the method comprising the steps of:
providing a saw guide including a distal end portion including a first hole for receiving a screw, a proximal end portion including a second hole for receiving a screw, and a center portion, the center portion including a first cutting plane and a second cutting plane, the position of the second cutting plane being adjustable relative to the first cutting plane;
placing a first screw through the first hole and tightening the first screw to secure the distal end portion to the bone;
placing a second screw through the second hole and tightening the second screw to secure the proximal end portion to the bone;
adjusting the second cutting plane relative to the first cutting plane to separate the first and second cutting planes by a desired distance;
making a first cut in the bone relative to the first cutting plane;
making a second cut in the bone relative to the second cutting plane; and
removing the portion of bone defined between the first and second cuts.

62. The method of claim 61, wherein the first hole is a first keyhole and the second hole is a second keyhole.

63. The method of claim 62, wherein the first placing step includes placing the first screw through the narrow portion of the first keyhole and tightening it and the second placing step includes placing the second screw through the narrow portion of the second keyhole and tightening it.

64. The method of claim 63, further comprising the step of, after making the second cut, removing the saw guide by loosening the first and second screws and sliding the body of the saw guide along a longitudinal axis of the body to align the heads of the first and second screws with the wider portions of the first and second keyhole and lifting the saw guide up and over the heads of the first and second screws.

65. The method of claim 64, wherein the first screw is an anchoring screw and the second screw is an advancement screw, the method further comprising the steps of:
after removing the saw guide, aligning wider openings of first and second keyholes in an osteotomy plate with the heads of the first and second screws, respectively;
placing the heads of the first and second screws through the wider openings of the first and second keyholes of the osteotomy plate; and
sliding the osteotomy plate along the longitudinal axis in a direction and amount opposite to that performed in the removal step, until the shaft of the first screw contacts the distal end of the narrow portion of the first keyhole of the osteotomy plate and tightening the first screw.

66. The method of claim 61, wherein the first cutting plane and the second cutting plane are oriented parallel to each other and perpendicular to a longitudinal axis through the body of the cutting guide.

67. The method of claim 61, wherein the first cutting plane and the second cutting plane are oriented parallel to each other and obliquely to a longitudinal axis through the body of the cutting guide.

68. The method of claim 67, wherein the cutting planes are oriented at a 45° angle to the longitudinal axis through the body of the cutting guide.

69. The saw guide of claim 61 wherein the second cutting plane is defined by an adjustable plate that is movable along the longitudinal axis of the saw guide, the adjustable plate including a threaded hole for engaging threads on a shaft of an adjustment screw.

70. The method of claim 69, wherein the adjusting step includes adjusting the adjustment screw to move the adjustable plate relative to the first cutting plane.
